(19) United States
(12) Patent Application Publication
(10) Pub. No.: US 2006/0036304 A1
(43) Pub. Date: Feb. 16, 2006

(54) Thermal garment system and method of using the same

(76) Inventors: Peter Cordani, Palm Beach Gardens, FL (US); Anne Cordani, Palm Beach Gardens, FL (US)

Correspondence Address: MCHALE & SLAVIN, P.A., 2855 PGA BLVD, PALM BEACH GARDENS, FL, 33410, US

(21) Appl. No.: 11/251,005

(22) Filed: Oct. 6, 2005

Related U.S. Application Data
(63) Continuation-in-part of application No. 10/001,047, filed on Nov. 1, 2001, now abandoned.

(51) Int. Cl. A61F 7/00

(52) U.S. Cl. 607/108; 607/112; 607/114

(57) ABSTRACT

The instant invention comprises a thermal garment system worn by a user to cool the user's upper torso and thereby reducing the incidence of heat stress related injuries. The garment comprising a plurality pockets integrally attached to adapted to receive and support at least one thermal pack therein. The thermal pack comprising a sealed container having flexible walls enclosing a thermal composition and a catalyst adapted to produce an endothermic reaction when desired. The pack includes at least one venting means therein so that the thermal container is more readily capable of being conformed to user's body. The present invention further includes a method for cooling a user's torso and/or body which includes placing the vest around a user's torso, activating the amount of thermal packs believed necessary to produce the desired amount of cooling, and placing the thermal packs into at least one pocket of the vest.

### CROSS REFERENCE TO RELATED APPLICATION

This application is a continuation-in-part of co-pending application Ser. No. 10/001,047, filed Nov. 1, 2001, the contents of which are herein incorporated by reference.

### FIELD OF THE INVENTION

This invention relates to a thermal garment system; particularly to a vest housing at least one cooling pack therein, the vest intimately conforming to the shape of the user's torso to prevent the occurrence of heat-related illness.

### BACKGROUND OF THE INVENTION

Heat stress refers to the combination of factors that increase the body's core temperature, such as: environmental conditions, clothing and metabolic rate. Heat stress disorders include: heat cramps, heat exhaustion-and heat stoke, with heat cramps being the least severe and heat stoke being potentially life-threatening.

When an individual's heat balance is compromised, the body will try to direct heat away from the core by increasing blood flow to the skin. When the body cannot sufficiently cool itself, injuries related to heat stress occur. Heat stress is often a significant risk for individuals who perform hard work in a hot and/or humid environments, for example, firefighters, police officers, military personnel, athletes, factory/plant workers (e.g., welders), construction workers (e.g., roofers), etc.

Often most heat related illness is treated after the individual's body has reached active hyperthermia. While some heat-exhausted individuals may be able to cool off enough on their own, it best to use external cooling methods to help. Numerous methods of rapidly cooling an individual's core body temperature have been effectively employed in the past. Some of these include: ice packs or towels, ice water immersion, whole-body liquid cooling garments, head cooling units, cool water spray, etc. The most effective placement of ice or cold packs has been found on the skin of the neck and underarms. This promotes rapid cooling of the blood. Few prior art devices or methods have attempted to prevent heat stress related illness before it occurs.

While some prior art garments do disclose the use of attachable thermal packs for engendering a change in the body temperature of the user, these thermal packs must be stored in a cooling chamber until such time as they need to be used. This severely limits the portability and practicality of such garments. Often, emergency stations and vehicles are limited in space and cannot afford to include cooling chambers as they take up valuable workspace and other resources.

What is needed is a cooling garment that will eliminate the problem of maintaining cold packs at the necessary temperature until needed. The instant device solves the aforementioned problems through the use of thermal appliances that nearly instantly create an endothermic reaction on demand, to transfer thermal energy to the body while inside the cooling garment.

Moreover, these thermal appliances can be made pliant and flexible by being able to evacuate the gaseous by-products always produced during the endothermic reaction. In prior devices, the gaseous by-produces become trapped in the pack rendering the thermal pack rigid and lacking in the ability to conform to the anatomy. In the extreme, the pack may rupture putting the reacting chemicals in direct contact with the patient's body. Moreover, the trapped gas may act as an insulator, which can inhibit effective thermal transfer to the user.

### DESCRIPTION OF THE PRIOR ART

U.S. Pat. No. 6,931,875 to Allen et al., discloses a basic vest with a plurality of pockets adapted to receive cooling packs therein for dissipating the cooling temperature thereto. The cooling packs must be initially placed in cooling chamber, such as freezer. It is in this manner that the cooling packs are cooled prior to being placed in the pockets of the vest. The reference fails to teach or suggest thermal appliances that are able to create an endothermic or exothermic reaction on demand.

U.S. Pat. No. 6,189,149 to Allen discloses a temperature change vest for fitting around person's torso and having a plurality of horizontally disposed pockets designed to house thermal packets therein. These thermal packets must be frozen or heated prior to use, making them less independent and instantly accessible than the instant invention. Again, this reference does not teach or suggest the use of packets able to create an instant endothermic or exothermic reaction.

U.S. Pat. No. 6,185,744 to Poholski discloses a personal thermal garment having a shell and thermal insert. Again the thermal insert must be preheated or precooled to transfer thermal energy to the user.

U.S. Pat. No. 5,604,144 to Simmons et al., discloses a garment that applies heat to the neck, head or torso with air-activated chemical heating packets. The reference fails to teach or suggest using a compliant container that creates a thermal reaction therein by mixing two or more chemical compounds when desired.

U.S. Pat. Nos. 5,484,448 and 5,146,625 both to Steele et al., disclose a cooling garment constructed and arranged to contain a plurality of cooling packs contained within respective elongated pockets. The cooling garment is particularly useful for the treatment of physiological conditions, including multiple sclerosis and other disorders. The cooling packs refer to ice packs containing ice or a gel mixture with high heat capacity that must first be frozen in a freezer. These references fail to teach a container that creates an endothermic reaction therein by mixing two or more chemical compounds on demand.

None of the aforementioned references, nor the combination thereof, teach or suggest a garment system capable of cooling a user's torso comprising a plurality pockets integrally attached to adapted to receive and support at least one thermal pack therein.

### SUMMARY OF THE INVENTION

The instant invention comprises a thermal garment system worn by a user to provide cooling of the user's upper torso comprising a plurality pockets integrally attached thereto and adapted to receive and support at least one thermal pack therein. The thermal pack comprising a sealed container having flexible walls enclosing a thermal composition and a catalyst adapted to produce an endothermic reaction.

The present method further includes a method for cooling a user's torso and/or body by placing the vest containing at least one thermal pack therein around an individual's torso, activating the appropriate amount of thermal packs necessary to produce the desired amount of cooling.

Accordingly, it is an objective of this invention to teach a garment in which the user is able to vary the amount of pockets and/or thermal packets, thus, the amount of thermal energy delivered to the body in proportion to the amount of heat stress.

Yet another objective of the instant invention is to provide a garment that will position at least one thermal packet close to the user's body and can be activated while the person is engaged in physical activity to help prevent heat related stress.

Another objective of the instant invention is to teach a garment capable of providing medical treatment to person having active hyperthermia.

Still another objective of the instant invention is to teach a garment that can be used by a variety of different sized users.

Yet another objective of the instant invention is to teach a cooling garment that can be activated even when worn under protective clothing, such as, a fire-retardant jacket.

Another objective of this invention is to teach a garment designed to carry and hold one or more thermal packs proximate the user's body at specific locations where blood vessels are close to the skin (e.g., under arms) to provide a more efficient transfer of thermal energy to the core of the body, thus, faster cooling.

Yet another objective of the instant invention is to provide a garment whereby the thermal packets, or the pockets themselves, maybe added or removed to increase or decrease the net weight of the garment as desired.

A further objective of this invention is to teach a garment capable of being worn by any person whose core body temperature may be potentially elevated by the milieu, such as: firefighters, police officers, military personnel, athletes, factory/plant workers (e.g., welders), construction workers (e.g., roofers), etc.

Another objective of this invention is to teach the thermal packs that do not need to be placed within a cooling chamber prior to use, thereby making it more portable, practical and instantly available for emergency situations.

### BRIEF DESCRIPTION OF THE DRAWINGS

FIG. 1 is a front perspective view of a thermal garment according to the invention worn by a user;

FIG. 2 is a back perspective view of the instant invention depicted in FIG. 1, showing the removable attachment of front panel and back panel along their corresponding shoulder straps;

FIG. 3 illustrates a view of the interior of the garment shown in FIG. 1, having a plurality of pockets integrally attached thereto;

FIG. 4 is cross-sectional view of a pocket of the instant garment with a thermal pack housed therein;

FIG. 5 shows a perspective of another embodiment of the thermal pack;

FIG. 6 shows a cross section of the one-way valve in the closed position, as seen along line A-A of FIG. 5; and

FIG. 7 shows a cross section of the one-way valve in the closed position, as seen along line A-A of FIG. 5.

### DETAILED DESCRIPTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representative basis for teaching one skilled in the art to various employ the present invention in virtually any appropriately detailed structure.

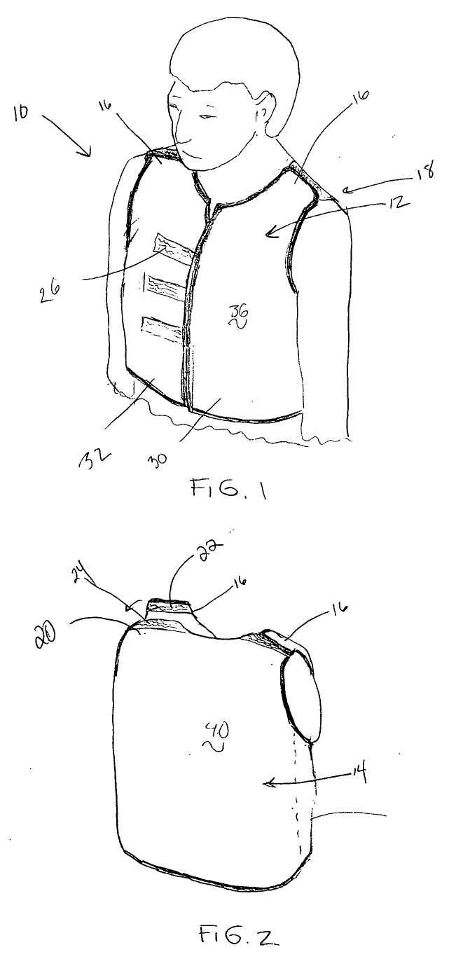

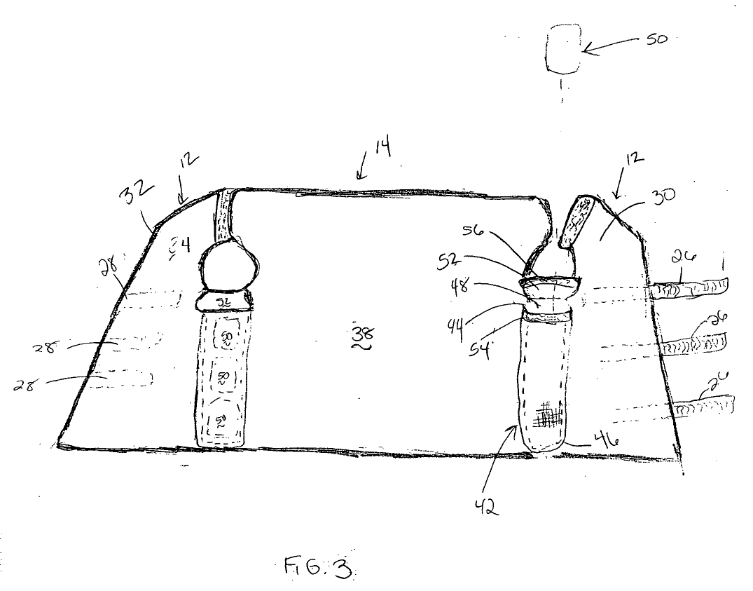

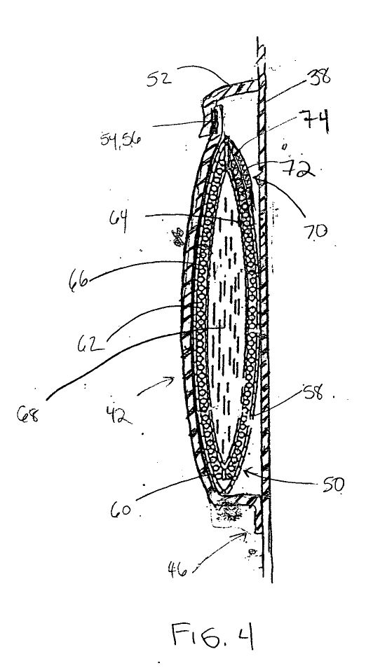

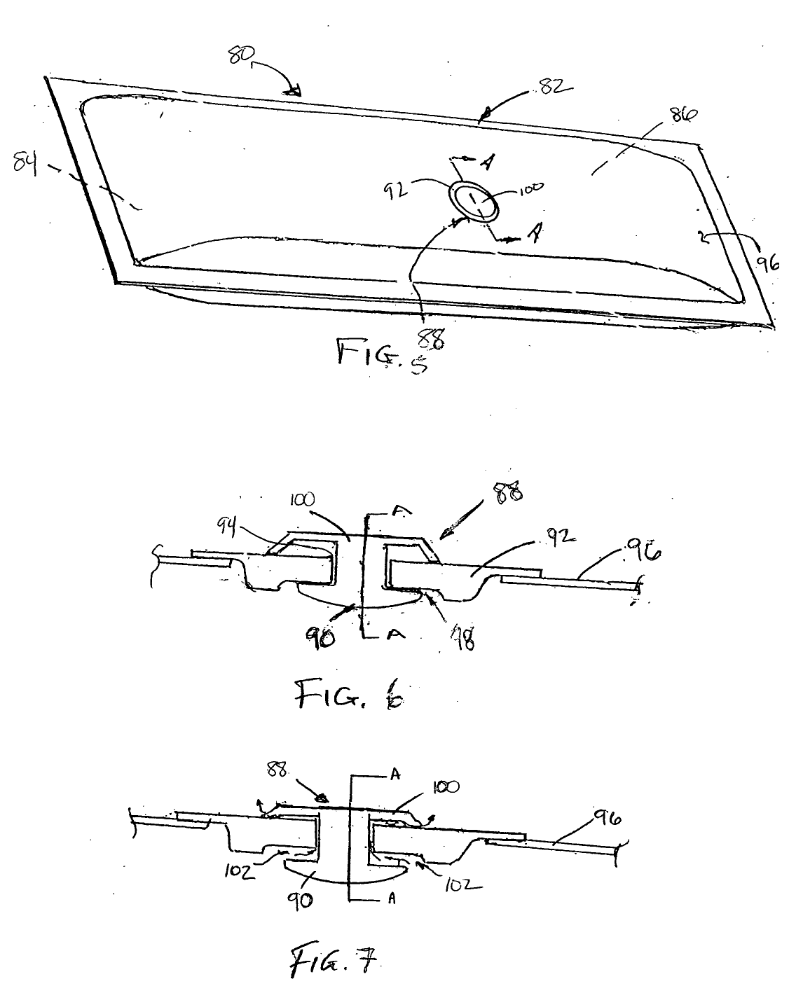

Referring now to FIGS. 1-7, wherein like elements are numbered consistently throughout, FIG. 1 illustrates an upper perspective view of the garment shown being worn on the torso of individual, generally referenced as 10. The vest comprises a front and back panel 12 and 14, respectively (FIG. 2). The front panel 12 having a interior surface 34 (FIG. 3), exterior surface 36 (FIG. 1) and a pair of upwardly extending shoulder straps 16 that lay snuggly against the shoulder portion 18 of the individual and works in conjunction with a corresponding pair of shoulder straps 20 integrally connected to the back panel portion 14, thereby providing a means for adjusting the size of the vest for a variety of different users.

In this regard, the pair shoulder straps on the front panel include a means of fastening that comprise a first portion 22 located along the upper part the shoulder straps (FIG. 2). Similarly, the means of fastening further comprises a second portion 24 provided along the upper portion of the pair of shoulder straps 20 on the back panel. In a preferred embodiment, the first and -second portion comprise hook or pile material (i.e., VELCRO) sewn on the material of the panels, however, any other substantially equivalent means of fastening the panels together could be used herein, including, snaps, hooks, zippers, or the like.

As seen in FIGS. 2 and 3, the back panel includes an interior and exterior surface 38, 40 and is integrally connected with the front panel 12 along the bottom portion thereof, i.e., under the armholes of the garment. The front and back panels may be connected by any means of attaching material known to the skilled artisan, such as,. sewing, adhesives, etc.

In a preferred embodiment, shown in FIGS. 1 and 3, the front panel comprises two laterally disposed panels 30, 32 which can be securely fastened together along the front of the torso of an individual using at least one attachment means depicted here as a plurality straps 26 integrally connected to either the panels 30, 32. As with the shoulder straps 16, 20, the attachment means includes a corresponding second attachment means 28 provided on the alternate front panel. In a preferred embodiment, the first and second attachment means 26, 28 may comprise corresponding hook or pile material (i.e., VELCRO). This allows the instant garment to be snugly fitted on users of various sizes. However, any other substantially equivalent means of attaching the front panels could be used herein without departing from the scope of the invention, including, snaps, hooks, zippers, or the like.

As shown in FIG. 3, the interior of the garment includes a plurality of integrally connected pockets 42 preferably sewn onto the interior surface 34, 38 of the either the front or back panels or at the bottom interface of the front and back panels. In a preferred embodiment, the garment is designed to hold one or more thermal packs proximate the user's body, preferably, along specific locations where blood vessels are close to the skin, such as under the user's arms to provide a more efficient transfer of thermal energy to the core of the body, thereby providing faster cooling. Although not shown herein, it is contemplated herein that the additional pockets 42 could be integrally connected to the exterior surfaces of the front and back panels 36, 40 to provide additional cooling and/or storage.

Each pocket has an open end 44 and a closed end 46 defining an inner cavity 48 adapted to receive and support at least one thermal pack therein. The upper peripheral the pocket opening 48 can include a flap of material designed to overlap and close the opening when desired. The peripheral edges of the flap and opening can include any corresponding fastening means known in the art, such as, hook/loop material 54, 56 (FIG. 3), snaps, buttons, zippers, etc.

In an alternative embodiment, the pockets 42 could be removably attached to the interior and/or exterior surface of the front and back panels by any suitable fastening means, such as hook and loop material (i.e. VELCO). This would provide an additional means for adjusting the amount of cooling desired and also provide a means to lessen the overall weight of the garment by removing any unused pockets, which is particularly advantageous to athletes using the instant device to regulate body temperature with the least amount of weight possible for maximum performance.

Referring now to FIG. 4, which illustrates a cross-sectional view of one embodiment of the pocket 42 containing a thermal pack 50 therein. The pack may be made in different shapes (e.g., rectangular, circular, cylindrical, etc) as a matter of choice and for special applications. The pack is made of an outer container having supple plastic film forming walls 58 and 60 sealed along the peripheral edges to form a liquid tight seal. The film may be single ply or co-extrusions of different polymers or laminated films having the same properties or different properties. The walls are formed of materials that will be impervious to each of the ingredients and to the resulting combination. The outer container may be made using a tubular plastic film that is sealed at the opposite ends. The plastic film may be thermoplastic and sealed by heat and pressure. If other plastic films are used, adhesives or solvents may form the seal.

As illustrated in FIG. 4, the outer container encloses at least one ingredient, a chemical compound 62, which will initiate a thermal reaction when mixed with another ingredient, in the form of a catalyst. Preferably, when the chemical compound is mixed with the catalyst, a gel-like substance is formed. The gel acts to prolong the thermal effects.

The outer container encloses another tube having opposing walls 64 and 66. The walls 64 and 66 are preferably a continuous plastic film sealed at the end. These films and seals may be formed in the same manner as the films and seals in the outer container. Superimposing film layers and sealing the superposed periphery may form the tube. The opposing walls 64 and 66 have a structure or are made from a material that will rupture before the outer container, when placed under a compressive load. This insures the integrity of the pack and prevents the thermal compound from coming into contact with the user.

The catalyst 68 for the thermal reaction is enclosed within the inner container until the inner container is ruptured. Once the thermal compound and catalyst come into contact with each other, the outer container is kneaded by the user to thoroughly mix the ingredients.

As the thermal reaction progresses, there is often generated a gaseous by-product. In a preferred embodiment, the volume of the gas can be released from the interior of the container through at least one venting means, shown here as an aperture 70 and porous membrane 72. The porous membrane is made from a material that will allow passage of gas, including any air trapped in the interior, but not liquid. The membrane spans the aperture 70 and has a continuous edge seal 74 joining the membrane to the container. Heat and pressure, solvent, or adhesives may form the edge seal 74. The material of the membrane may be a semipermeable or a microporous non-woven material. Such releasing of gaseous by-products creates a more flexible container that will conform to the user's body and less likely to burst. Moreover, the trapped gas tends to create a layer of insulation within the container and thus raises the insulation value of the same. This tends to inhibit the effective transfer of energy to the user.

The venting means of FIG. 4 may be a hydrophobic microporous nonwoven plastic material such as TYVEK made by DuPont Co. The vent material will allow gas to pass through, while repelling liquid.

FIGS. 5-7 illustrate an alternative embodiment of the thermal pack 80. This thermal pack 80 has a peripheral seal 82 and encloses a catalyst 84 inside the bag which is separated from a thermal composition 86 by a frangible wall. In this embodiment the venting means is a one-way valve 88 that has an annular portion 90 on the internal end of a valve body that reciprocates through a collar 92 sealed into an aperture 94 in the thermal pack wall 96. The annular portion is seated in an annular recess 98. It is contemplated herein that the thermal pack shown in FIGS. 5-7 would comprise a plurality of one-way valves to ensure venting of gaseous by-products when the pack is virtually any position.

As depicted in FIG. 6, the pressure of the gas by-product of the thermal reaction caused by mixing the catalyst and the thermal composition will act against the annular valve 90 to seat the valve in the recess 98, thereby closing the one-way valve. The user can depress the valve actuator 100 to overcome the gas pressure closing the valve. This causes the valve body to move and displace the annular portion 90 thereby creating a vent passageway 102. The gaseous by-product and any other trapped gases are released through the passageway.

In one example of the thermal pack, the outer container may be fabricated from polyethylene film, alone, or laminated with other materials. The thermal compound may be ammonium nitrate, either alone or mixed with other chemicals, disposed inside the outer container. Preferably, the ammonium nitrate is in dry particle form.

The inner container may be made of the same plastic composition as the outer container. The inner container may have a weakened portion of a wall or the container may be of a thinner film. The catalyst, enclosed in the inner container, is water alone, or mixed with other chemicals.

One advantage of the instant invention is that the thermal pack may be stored indefinitely until the ingredients are mixed. Moreover, the thermal pack need not stored in a cooling chamber (e.g., freezer) prior to use.

To provide instant cold treatment to a user, the compliant envelope is squeezed or put under compressive pressure to rupture the inner tube. The container is then kneaded to thoroughly mix the chemicals and produce a temperature change. As the chemical endothermic reaction proceeds, gas evolves and escapes from the container through the semi-permeable vent leaving the gel product. The cold compliant container is then placed within one of the pockets of the garment.

One of more thermal packs may be inserted into an insulating sleeved prior to placement into the pocket, such that the amount of thermal energy delivered by the cooling pack may be attenuated. The insulating sleeve may be composed of any material known in the art (i.e., THINSULATE) to reduce the incidence of discomfort and/or possible frostbite.

As to the manner of usage and operation, the instant thermal garment system can be worn by a user to regulate body temperature prior to the incidence of heat stress or could be applied to a person in active hyperthermia to lower the body temperature in an emergency situation.

For example, in the former instance, the garment containing a sufficient number of thermal packs in the pockets is placed in thermal communication with the user's torso with a minimal mount of clothing therebetween, preferably for instance, a firefighter would wear the garment under any protective clothing (fire-retardant jacket).

The front and back panels of the vest are securely fastened together along the shoulder portion. Similarly, the front panels are securely attached together by the plurality straps to snuggly fit the torso of the individual. Once the user's feels overheated, a user applies exterior pressure to protective clothing or directly to the vest where pockets containing at least one thermal pack are located. This can be as easy as a downward motion of the arms/elbow over the pockets with sufficient compressive force to rupture the frangible partition that separates the thermal composition and the catalyst of the pack, such that the thermal composition and the catalyst mix thoroughly to initiate an endothermic reaction and thereby provide instant cooling to the user. The venting means of the container will preclude the container from bursting upon application of the exterior activating pressure.

It is contemplated herein that the vest may be formed of any material desired and be made in any color including, albeit not limited to, safety orange, red, camouflage, etc.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A thermal garment system worn by a user to provide cooling of the user's upper torso comprising, in combination:
a vest having front and back panels to substantially cover and conform to the chest and back of said user's torso, respectively, said front and said back panels each having an interior surface, exterior surface, an upper and lower portion, wherein said upper portions of each said panels include a integrally attached shoulder straps, to removably attach said front and back portions together and define an opening through which said user's head extends, when worn;
a plurality pockets integrally attached to at least one of said panels, each of said pockets having an open end and a closed end defining an inner cavity adapted to receive and support at least one thermal pack therein;
said thermal pack comprising a sealed container having flexible walls, said container having a frangible partition separating said thermal composition and said catalyst, said thermal composition adapted to produce an endothermic reaction when mixed with said catalyst.

2. The thermal garment system as set forth in claim 1, wherein said container includes at least one venting means disposed within said flexible walls to provide a outlet for any gaseous by-products produced by said endothermic reaction, whereby said container may conform to said user.

3. The thermal garment system as set forth in claim 2, wherein said venting means comprises an aperture defined by a continuous edge, a hydrophobic membrane spanning said aperture and sealed to said edge, said membrane adapted to pass any gaseous by-products produced by said endothermic reaction while blocking liquid, whereby said gaseous by-products exit said container through said membrane so that said thermal container is capable of being conformed to user's body.

4. The thermal garment system as set forth in claim 2, wherein said venting means comprises at least one one-way valve mounted with said flexible walls, said one-way valve being manually operable, whereby any said gaseous by-products exit said container through said valve so that said thermal container is capable of being conformed to user's body.

5. The thermal garment system as set forth in claim 1, wherein said thermal composition is a liquid.

6. The thermal garment system as set forth in claim 1, wherein said thermal composition is a gel.

7. The thermal garment system as set forth in claim 1, wherein said partition is an envelope containing one of said thermal composition or said catalyst, said envelope having walls formed of a plastic film, said walls adapted to rupture in response to compression.

8. The thermal garment system as set forth in claim 7, wherein said catalyst is water.

9. The thermal garment system as set forth in claim 7, wherein said thermal composition includes ammonium nitrate.

10. The thermal garment system as set forth in claim 1, wherein said plurality pockets are permanently attached to said interior surface of one of said first or second panels.

11. The thermal garment system as set forth in claim 1, wherein said plurality pockets are removably attached to said interior surface of one of said first or second panels.

12. The thermal garment system as set forth in claim 1, wherein said front and back pair of said shoulder straps are removably together by corresponding securing means selected from the group consisting essentially of: hook and loop material, zippers, snap buttons, hooks or the like.

13. A method of reducing the incidence of heat stress related injuries by preventing or lowering elevated body temperature in a user through a thermal garment system, comprising:
placing said thermal garment system comprising a vest having front and back panels to substantially cover the front and back of a user's torso, respectively, said front and said back panels having an interior surface, exterior surface, an upper and lower portion, wherein said upper portions of said panels include integrally attached shoulder straps to removably attach said front and back portions together and define an opening through which said user's head extends when worn, said vest further including a plurality pockets integrally attached to at least one of said first and second panels, each of said pockets adapted to receive and support at least one thermal pack therein;
initiate a endothermic reaction in at least one thermal pack, said thermal pack comprising a sealed container having flexible walls having a frangible partition separating a thermal composition and a catalyst, whereby said frangible partition is ruptured and said thermal composition and said catalyst kneaded to thoroughly mix the ingredients to initiate said endothermic reaction.

14. The thermal garment system as set forth in claim 13, wherein said container includes at least one venting means disposed within said flexible walls to provide a vent for any gaseous by-products produced by said endothermic reaction, whereby said container may conform to said user.

15. The thermal garment system as set forth in claim 14, wherein said venting means comprises an aperture defined by a continuous edge, a hydrophobic membrane spanning said aperture and sealed to said edge, said membrane adapted to pass any gaseous by-products produced but block liquid, whereby said gaseous by-products exit said container through said membrane so that said container is made more flexible and able to more readily conform to user's body.

16. The thermal garment system as set forth in claim 14, wherein said venting means comprises at least one one-way valve mounted with said flexible walls, whereby any said gaseous by-products exit said container through said valve, whereby said thermal container is made more flexible and able to more readily conform to user's body.

17. The thermal garment system as set forth in claim 13, wherein said thermal composition is a liquid.

18. The thermal garment system as set forth in claim 13, wherein said thermal composition is a gel.

19. The thermal garment system as set forth in claim 13, wherein said partition is an envelope containing one of said thermal composition or said catalyst, said envelope having walls formed of a plastic film, said walls adapted to rupture in response to compression.

20. The thermal garment system as set forth in claim 13, wherein said catalyst is water.

21. The thermal garment system as set forth in claim 13, wherein said thermal composition includes ammonium nitrate.

22. The thermal garment system as set forth in claim 13, wherein said plurality pockets are permanently attached to said interior surface of one of said first or second panels.

23. The thermal garment system as set forth in claim 13, wherein said plurality pockets are removably attached to said interior surface of one of said first or second panels.

24. The thermal garment system as set forth in claim 13, wherein said front and back pair of said shoulder straps are removably together by corresponding securing means selected from the group consisting essentially of: hook and loop material, zippers, snap buttons, hooks or the like.
